(19) United States
(12) Patent Application Publication
(10) Pub. No.: US 2023/0010961 A1
(43) Pub. Date: Jan. 12, 2023

(54) ADJUSTABLE COVER FOR AN AUTOMATED MEDICAL DEVICE

(71) Applicant: XACT ROBOTICS LTD., Caesarea (IL)

(72) Inventors: Eli LEVIN, Atlit (IL); Ben GALILI, Atlit (IL); Yoav TIKOCHINSKY, Tel Aviv (IL); Edna GADON, Tel Aviv (IL); Eran ZUR, Karkur (IL); Idan ZILZER, Kibbutz Dan (IL)

(21) Appl. No.: 17/778,856

(22) PCT Filed: Nov. 26, 2020
(86) PCT No.: PCT/IL2020/051220

§ 371 Date: May 22, 2022

Related U.S. Application Data
(60) Provisional application No. 62/942,114, filed on Nov. 30, 2019.

(51) Int. Cl. A61B 50/30; A61B 90/11

(52) U.S. Cl. CPC A61B 50/30; A61B 90/11

(57) ABSTRACT

Provided are adjustable protective covers for automated medical devices, that protect inner and/or outer components of the medical device, while maintaining the functionality of the automated device.

### FIELD OF THE INVENTION

The present invention relates to the field of protective covers for automated medical devices, specifically to adjustable protective covers for automated medical devices, and more specifically to adjustable protective covers that shield inner and/or outer components of the automated medical device while maintaining the functionality of the medical device.

### BACKGROUND

Many routine treatments employed in modern clinical practice involve percutaneous insertion of medical tools/instruments, such as needles and catheters, for biopsy, drug delivery and other diagnostic and therapeutic procedures. The aim of an insertion procedure is to place the tip of an appropriate medical tool safely and accurately in a target region, which could be a lesion, tumor, organ or vessel. Examples of treatments requiring insertion of such medical tools include vaccinations, blood/fluid sampling, regional anesthesia, tissue biopsy, catheter insertion, cryogenic ablation, electrolytic ablation, brachytherapy, neurosurgery, deep brain stimulation and various minimally invasive surgeries.

The guidance and steering of medical tools, such as needles, in soft tissue is a complicated task that requires good three-dimensional coordination, knowledge of the patient's anatomy and a high level of experience, thus the use of automated (e.g., robotic) systems for performing these functions has been increasingly growing in recent years.

When a medical procedure is being performed, it is of utmost importance to maintain a sterile and clean environment in the procedure room. If a non-sterile component comes in contact with the patient's body during the procedure, the patient may be infected with a variety of bacteria and other contaminants, which may be hazardous to his/her health. When body-mountable devices, or devices which are positioned in close proximity to the patient's body, are being used for the medical procedure, the patient's blood and/or other bodily fluids and tissues may soil the device during the procedure. If the automated device is reusable, at least in part, such that the same automated device (or components thereof) is to be used for performing a medical procedure on another patient, cross-contamination between patients may occur.

Thus, if the automated device is reusable, at least in part, it should preferably undergo sterilization between consecutive procedures. However, automated devices typically include electromechanical components, such as motors, sensors and electrical wires, which cannot undergo sterilization, therefore, it is of importance to ensure that the non-sterile parts of the device are maintained covered throughout the entire medical procedure, such that none of the non-sterile components of the device can contact the patient's body.

Moreover, it is of importance to ensure that the driving and/or steering mechanisms of the device are maintained covered also during storage and between consecutive procedures, so as to prevent dust/dirt from entering these mechanisms and compromising the integrity and functionality and/or efficiency and/or accuracy of the automated device.

Further, it is of importance to ensure that covering the non-sterile parts of the device does not compromise the operation of the device during use, in particular, does not limit the translation and rotation ranges of the driving and/or steering mechanisms and/or compromise the stability of the device.

Thus, there is a need for an adjustable cover for an automated medical device that can effectively protect the elements/components of the automated medical device (such as various elements of the driving and/or steering mechanisms), and prevent/minimize the risk of cross-contamination between patients, while maintaining an efficient operation thereof.

### SUMMARY

The present disclosure is directed to various adjustable protective covers of an automated medical device, such as an automated robot for insertion of a medical tool into a subject body, wherein the adjustable protective cover is compatible with the device components and allows protecting the internal and/or external (exposed) components of the device on the one hand, without compromising the operation capabilities of the automated medical device.

According to some embodiments, provided herein are advantageous adjustable protecting covers for automated medical devices, such as automated insertion devices utilized for automated insertion of a medical instrument into a target within the body of a subject, wherein the adjustable cover allows protection of inner and/or outer components of the device (for example, from bodily fluids, and other environmental deposits, such as, airborne bacteria, humidity, and the like) without compromising the movement of the driving and/or steering elements of the device, i.e. allowing full range of operation of the device.

According to some embodiments, the advantageous adjustable cover allows maintaining the device's performance, including workspace, accuracy, functionality, and the like. In some embodiments, the adjustable cover allows protecting and, in some instances, further partial sealing of the inner components of the medical device, from external environment. In some embodiments, the adjustable cover may allow protecting external components of the device, such as, various parts/elements of the driving and/or steering mechanism.

According to some embodiments, the adjustable cover may be made of one unit or may include one or more covering elements that may change their shape, size and/or relative location, so as to allow adjustable covering of the device and components thereof, while allowing full maneuverability of the device. In some embodiments, the adjustable cover may be part of a fixed/stationary housing of the device. The housing (also referred to as “stationary cover portion” or “fixed cover portion”) may be rigid or semi-flexible, and it may be attached to, or an integral part of, a base of the device. In some embodiments, the adjustable cover may be permanently or transiently attached to the housing of the device. In some embodiments, the adjustable cover may be rigid, semi-rigid, flexible, elastic, or combinations thereof. In some embodiments, the adjustable cover (and the cover elements, if more than one element is used) may be made of any suitable material, such as, rubber, plastic, metal, aluminum, and the like. In some embodiments, the adjustable cover (and the cover elements, if more than one element is used) may have any desired shape, size, thickness, elasticity and/or flexibility. In some embodiments, in instances where the adjustable cover is comprised of discrete cover elements, the cover elements may be identical, similar or different from each other with respect to size, shape, thickness, flexibility, composition, and the like.

According to some embodiments, the adjustable cover is reusable. In some embodiments, the adjustable cover is disposable. In some embodiments, the adjustable cover is integrally formed with the housing of the device. In some embodiments, the adjustable cover is mounted, attached or placed on the housing of the device.

According to some embodiments, the adjustable cover further allows serviceability of inner components of the medical device.

According to some embodiments, the adjustable cover may be used in addition to a sterile drape.

According to some embodiments, the medical tool is configured for insertion into a body of a subject. The medical tool may include one or more of: a needle, an introducer, a catheter, a cannula, a probe, a port, an electrode rod, a surgical tool and a fluid delivery tool.

According to some embodiments, the medical device is configured to be mounted on the body of the subject.

According to some embodiments, the medical device is configured to be positioned in close proximity to the subject's body, e.g., using a dedicated arm or carriage.

According to some embodiments, there is provided an adjustable cover for a medical device, the adjustable cover includes:

one or more stationary cover portions; and

one or more moveable cover portions having a plurality of moveable cover elements configured for movement relative to each other and relative to the one or more stationary cover portions,

wherein at least one of the plurality of moveable cover elements is configured to move relative to at least one of: (i) an adjacent moveable cover element of the plurality of moveable cover elements and (ii) the one or more stationary cover portions, as one or more components of a driving mechanism and/or a steering mechanism of the medical device move, such that both coverage and proper operation of the driving mechanism and/or the steering mechanism are maintained.

According to some embodiments, at least two of the plurality of moveable cover elements at least partially overlap.

According to some embodiments, the one or more stationary cover portions may include one or more openings and the plurality of moveable cover portions may be disposed within the one or more openings.

According to some embodiments, at least one opening of the one or more openings is a top opening located at a top surface of at least one of the one or more stationary cover portions.

According to some embodiments, at least one opening of the one or more openings is a front opening located at a front surface of at least one of the one or more stationary cover portions.

According to some embodiments, the one or more moveable cover portions may include a top moveable cover portion disposed within the top opening, and a front moveable cover portion disposed within the front opening, the front moveable cover portion being substantially perpendicular to the top moveable cover portion.

According to some embodiments, at least one opening of the one or more openings is a side opening located at a side surface of at least one of the one or more stationary cover portions.

According to some embodiments, the plurality of moveable cover elements are configured to slide one over the other.

According to some embodiments, each moveable cover element of the plurality of moveable cover elements is configured as a flat frame.

According to some embodiments, the one or more stationary cover portions may include one or more guides disposed on an inner surface thereof, the one or more guides being configured to guide the sliding movement of the plurality of moveable cover elements.

According to some embodiments, each of the one or more guides may include one or more rails configured to receive an end of at least one of the plurality of moveable cover elements.

According to some embodiments, each moveable cover element of the plurality of moveable cover elements is configured to be supported by two rails of oppositely disposed two guides of the one or more guides.

According to some embodiments, the one or more rails of the one or more guides are arranged in a crosshatch pattern.

According to some embodiments, the adjustable cover may further include one or more bearings configured to facilitate the sliding movement of the plurality of moveable cover elements, wherein the one or more bearings are positioned on at least one of the one or more stationary cover portions and at least one of the plurality of moveable cover elements.

According to some embodiments, the one or more moveable cover portions is configured to collapse or expand, according to the movement of the one or more components of the driving mechanism and/or the steering mechanism.

According to some embodiments, the plurality of moveable cover elements are configured as annular elements.

According to some embodiments, each of at least several of the annular elements is configured such that the annular element is smaller than a first adjacent annular element and larger than a second adjacent annular element.

According to some embodiments, the movement of the at least one of the plurality of moveable cover elements relative to an adjacent moveable cover element includes one or more of: (i) the annular element being partially inserted into or partially extended out of the first adjacent annular element, and (ii) the second adjacent annular element being partially inserted into or partially extended out of the annular element.

According to some embodiments, at least one of the one or more moveable cover portions is configured to be inserted, at least in part, into at least one of the one or more stationary cover portions.

According to some embodiments, the adjustable cover may further include at least one sealing layer disposed between at least two of the plurality of moveable cover elements.

According to some embodiments, at least one of the one or more stationary cover portions is configured for attachment to a base of the medical device.

According to some embodiments, the at least one of the one or more moveable cover portions is moveable by the movement of the one or more components of the driving mechanism and/or the steering mechanism.

According to some embodiments, the one or more moveable cover portions are rigid elements, flexible elements, semi-flexible elements, or a combination thereof.

According to some embodiments, the one or more moveable cover portions may be made of metal, plastic, rubber, silicone, aluminum, or any combination thereof.

According to some embodiments, the plurality of moveable cover elements are similar in size, shape and/or composition.

According to some embodiments, the plurality of moveable cover elements are different in size, shape and/or composition.

According to some embodiments, the adjustable cover is configured to cover inner components of the medical device.

According to some embodiments, the adjustable cover is configured to cover external elements of the medical device.

According to some embodiments, the medical device is an automated device comprising at least four degrees of freedom (DOF).

According to some embodiments, the medical device is an automated insertion device configured to insert a medical instrument into a body of a subject.

According to some embodiments, the adjustable cover is configured to at least partially cover and protect portions of the medical device from environmental deposits comprising one or more of: biological fluids, airborne bacteria, humidity, dust, dirt or any combination thereof.

According to some embodiments, there is provide an adjustable cover for a medical device, the adjustable cover includes:

one or more stationary cover portions, at least one of the one or more cover portions having one or more openings; and

one or more moveable cover portions disposed within the one or more openings and having a plurality of partially overlapping moveable cover elements, the moveable cover elements being configured for movement relative to each other and relative to the one or more stationary cover portions,

wherein at least one of the plurality of moveable cover elements is configured to move relative to at least one of: (i) an adjacent moveable cover element of the plurality of moveable cover elements and (ii) the one or more stationary cover portions, as one or more components of a steering mechanism of the medical device move, such that both coverage and proper operation of the steering mechanism are maintained.

According to some embodiments, each moveable cover element of the plurality of moveable cover elements is configured as a flat frame.

According to some embodiments, the one or more openings include a top opening located at a top surface of at least one of the one or more stationary cover portions.

According to some embodiments, the one or more openings further include a front opening located at a front surface of the at least one of the one or more stationary cover portions.

According to some embodiments, the one or more moveable cover portions include a top moveable cover portion disposed within the top opening, and a front moveable cover portion disposed within the front opening.

According to some embodiments, the top and front moveable cover portions are configured such that the movement of the one or more components of the steering mechanism of the medical device results in simultaneous movement of at least one moveable cover element of the top moveable cover portion and at least one moveable cover element of the front moveable cover portion.

According to some embodiments, the one or more stationary cover portions includes one or more guides disposed on an inner surface thereof, the one or more guides being configured to guide the movement of the plurality of moveable cover elements.

According to some embodiments, each of the one or more guides includes one or more rails configured to receive an end of at least one of the plurality of moveable cover elements.

According to some embodiments, there is provided an adjustable cover for a medical device, the adjustable cover includes:

one or more stationary cover portions; and

one or more moveable cover portions configured to collapse or expand, according to movement of a steering mechanism of the medical device, the one or more moveable cover portions having a plurality of partially overlapping annular elements,

wherein at least one of the plurality of annular elements is configured to move relative to at least one of: (i) an adjacent annular element of the plurality of annular elements and (ii) the one or more stationary cover portions, as one or more components of the steering mechanism moves, such that both coverage and proper operation of the steering mechanism are maintained.

According to some embodiments, each of at least several of the annular elements is configured such that the annular element is smaller than a first adjacent annular element and larger than a second adjacent annular element.

According to some embodiments, the movement of the at least one of the plurality of annular elements relative to an adjacent annular element includes the annular element being partially inserted into or partially extended out of the first adjacent annular element.

According to some embodiments, the annular elements include constraining members configured to limit the movement of the annular elements relative to each other.

According to some embodiments, the constraining members extend along a circumference of the annular elements.

According to some embodiments, at least one of the one or more moveable cover portions is configured to be inserted, at least in part, into at least one of the one or more stationary cover portions.

According to some embodiments, at least one of the one or more stationary cover portions is configured for attachment to a base of the medical device.

According to some embodiments, there is provided an automated medical device for insertion of a medical instrument into a body of a subject, the device includes: a steering mechanism comprising one or more actuators configured for inserting and steering the medical instrument into and within the body of the patient; and an adjustable cover as disclosed herein.

According to some embodiments, there is provided an automated medical device for insertion of a medical instrument into a body of a subject, the device includes:

a steering mechanism comprising one or more actuators configured for inserting and steering the medical instrument into and within the body of the patient; and

an adjustable cover having one or more stationary cover portions, and one or more moveable cover portions having a plurality of moveable cover elements configured for movement relative to each other and relative to the one or more stationary cover portions, wherein at least one of the plurality of moveable cover elements is configured to move relative to at least one of: (i) an adjacent moveable cover element of the plurality of moveable cover elements and (ii) the one or more stationary cover portions, as one or more components of a driving mechanism and/or a steering mechanism of the medical device move, such that both coverage and proper operation of the driving mechanism and/or the steering mechanism are maintained.

According to some embodiments, the steering mechanism of the automated medical device includes an inner portion and one or more external portions. According to some embodiments, the one or more external portions includes a top portion having one or more moveable arms, and wherein the inner portion of the steering mechanism is covered by the one or more moveable cover portions and the top portion is positioned externally to the one or more moveable cover portions.

According to some embodiments, there is provided an automated medical device for insertion of a medical instrument into a body of a subject, the device includes:

a steering mechanism having one or more actuators configured for inserting and steering the medical instrument into and within the body of the patient; and

an adjustable cover which includes one or more stationary cover portions, at least one of the one or more cover portions having one or more openings, and one or more moveable cover portions disposed within the one or more openings and having a plurality of partially overlapping moveable cover elements, the moveable cover elements being configured for movement relative to each other and relative to the one or more stationary cover portions, wherein at least one of the plurality of moveable cover elements is configured to move relative to at least one of: (i) an adjacent moveable cover element of the plurality of moveable cover elements and (ii) the one or more stationary cover portions, as one or more components of a steering mechanism of the medical device move, such that both coverage and proper operation of the steering mechanism are maintained.

According to some embodiments, there is provided an automated medical device for insertion of a medical instrument into a body of a subject, the device includes:

a steering mechanism comprising one or more actuators configured for inserting and steering the medical instrument into and within the body of the patient; and

an adjustable cover as disclosed herein, for example, an adjustable device which includes one or more stationary cover portions, and one or more moveable cover portions configured to collapse or expand, according to movement of a steering mechanism of the medical device, the one or more moveable cover portions having a plurality of partially overlapping annular elements, wherein at least one of the plurality of annular elements is configured to move relative to at least one of: (i) an adjacent annular element of the plurality of annular elements and (ii) the one or more stationary cover portions, as one or more components of the steering mechanism moves, such that both coverage and proper operation of the steering mechanism are maintained.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

Some exemplary implementations of the devices, systems and methods of the present disclosure are described with reference to the accompanying drawings. In the drawings, like reference numbers indicate identical or substantially similar elements.

FIG. 1 shows a schematic top view of portions of an exemplary medical device for inserting and steering a medical tool in a body of a subject, according to some embodiments;

FIGS. 2A-2D show schematic top (FIGS. 2A-2B) and side (FIGS. 2C-2D) views of exemplary optional movements of the steering mechanism of the medical device, according to some embodiments;

FIG. 3 shows a schematic illustration of a medical device for inserting and steering a medical tool, having an adjustable protective cover, according to some embodiments;

FIGS. 4A-4D show top views of an adjustable protective cover comprised of moveable cover portions, at various positions, according to some embodiments;

FIG. 5 shows a cross-sectional view of the external housing and the adjustable top protective cover of FIGS. 4A-4D, according to some embodiments;

FIG. 6 shows a schematic view of a medical device having a top adjustable cover and a front adjustable cover, according to some embodiments;

FIGS. 7A-7B show perspective views of the top and front adjustable protective covers of FIG. 6, at various positions, according to some embodiments;

FIG. 8 shows a bottom view of the top portion of the external casing and the adjustable protective cover of FIGS. 6 and 7A-7B, according to some embodiments;

FIGS. 9A-9B show partial perspective bottom views of the top portion of the external casing and the adjustable protective cover of FIGS. 6-8, according to some embodiments;

FIG. 10 shows schematic illustration of a medical device having a flexible top protective cover, according to some embodiments;

FIGS. 11A-11C show schematic top views of various flexible top protective covers, according to some embodiments;

FIGS. 12A-12C show schematic views of a medical device having a flexible adjustable protective cover, according to some embodiments;

FIGS. 13A-13D show schematic views of a medical device having an exemplary adjustable protective cover comprising a plurality of partially overlapping elements, according to some embodiments. FIG. 13A shows a perspective view of the medical device; FIG. 13B shows a side view of the medical device; FIG. 13C shows a top view of the medical device;

FIG. 13D shows a rear view of medical device;

FIGS. 14A-14B show schematic top views of the medical device of FIGS. 13A-13D, with the adjustable protective cover in collapsed (FIG. 14A) and extended (FIG. 14B) positions, according to some embodiments;

FIGS. 15A-15E show schematic views of a medical device having another exemplary adjustable protective cover comprising a plurality of partially overlapping elements, according to some embodiments.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

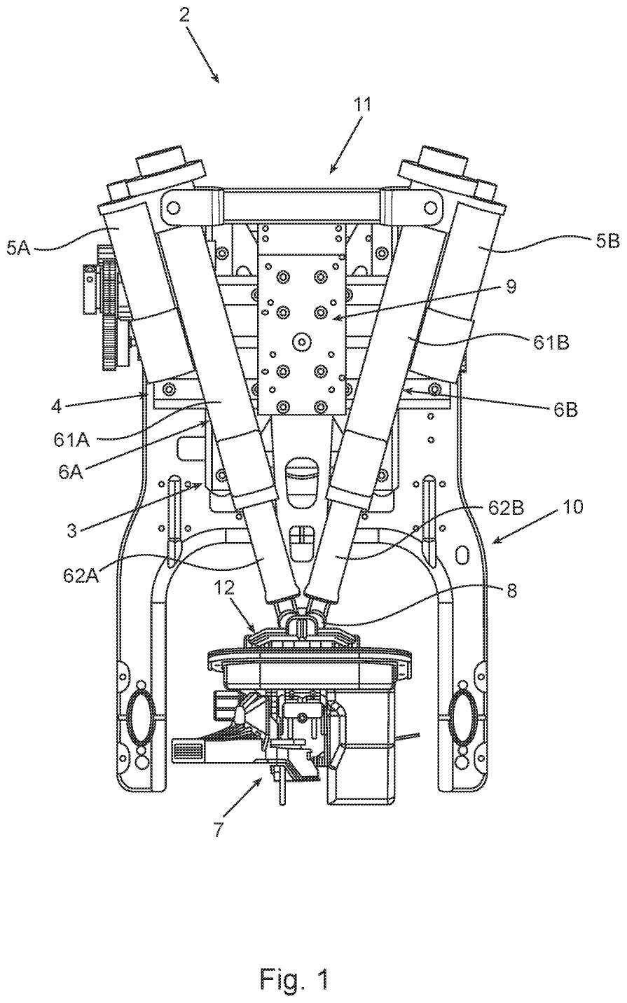

Reference is made to FIG. 1A, which shows a schematic top view of portions of an exemplary medical device for inserting and steering a medical tool in a body of a subject, according to some embodiments. As shown in FIG. 1A, the device 2 may include a base 10, a steering mechanism and an insertion mechanism. In some embodiments, the base 10 may be configured for securing to the subject's body, either directly or using a mounting apparatus, such as the mounting base disclosed in co-owned U.S. Patent Application Publication No. 2019/125,397, to Arnold et al, or the attachment frame disclosed in co-owned International Patent Application Publication No. WO 2019/234748, to Galili et al, both of which are incorporated herein by reference in their entireties. In some embodiments, the base 10 may be configured for coupling/attaching to a dedicated arm (stationary, robotic or semi-robotic) which can be secured to the subject's bed or to a cart positioned adjacent the subject's bed or to an imaging device (if such is used), and positioned, using the dedicated arm, on the subject's body or in close proximity thereto, as described, for example, in U.S. Pat. Nos. 10,507,067 and 10,639,107, both to Glozman et al, and both incorporated herein by reference in their entireties.

The steering mechanism may include at least one moveable platform and at least one moveable arm, configured to allow or control movement of an end effector (also referred to as “control head”), at any one of desired movement angles or axis. As shown in FIG. 1A, the device 2 may include a stationary platform 3, a first moveable platform 4, a second moveable platform 9 and two moveable arms 6A and 6B coupled to the second moveable platform 9, as disclosed, for example, in co-owned U.S. Patent Application Publication No. 2019/290,372, to Arnold et al, which is incorporated herein by reference in its entirety. The moveable arms 6A and 6B may be configured as piston mechanisms that may have one or more common connecting regions and/or joints (such as, connecting region 11 and joint 8 which connects the movable arms to the end effector 12, either directly or via a gimbal (not shown)). In some embodiments, each piston mechanism may include: a cylinder 61A and 61B, a piston 62A and 62B, at least a portion of which is being positioned within the cylinder, and a driving mechanism configured to controllably propel the piston in and out of the cylinder (for example, utilizing a motor, shown as motors 5A and 5B). To the end of the control head 12, a suitable medical instrument (not shown) may be connected, either directly or by means of a suitable insertion module 7, such as the insertion module disclosed in co-owned U.S. Patent Application Publication No. 2017/258,489, to Galili et al, which is incorporated herein by reference in its entirety. In some embodiments, the device's insertion mechanism (not shown) may be coupled to the control head 12. In some embodiments, a first portion of the device's insertion mechanism may be coupled to the control head 12, and a second portion of the device's insertion mechanism may be coupled to the insertion module 7, such that the insertion mechanism becomes operable only upon connection of the insertion module 7 to the control head 12. In the latter cases, the insertion module may be coupled to the control head directly, or a drape adaptor of a sterile drape may be attached first to the control head, and the insertion module may then be connected to the drape adaptor. An exemplary drape adaptor was disclosed in co-owned U.S. Patent Application Publication No. 2020/170,740, to Galili et al, which is incorporated herein by reference in its entirety.

The medical instrument may be any suitable instrument capable of being inserted and steered within the body of the subject, to reach a designated target, wherein the control of the operation and movement of the medical instrument is effected by the control head 12. For example, the medical instrument may be selected from, but not limited to: needle, probe (e.g., an ablation probe), port, introducer, catheter (such as a drainage needle catheter), cannula, surgical tool, fluid delivery tool, or any other suitable insertable tool

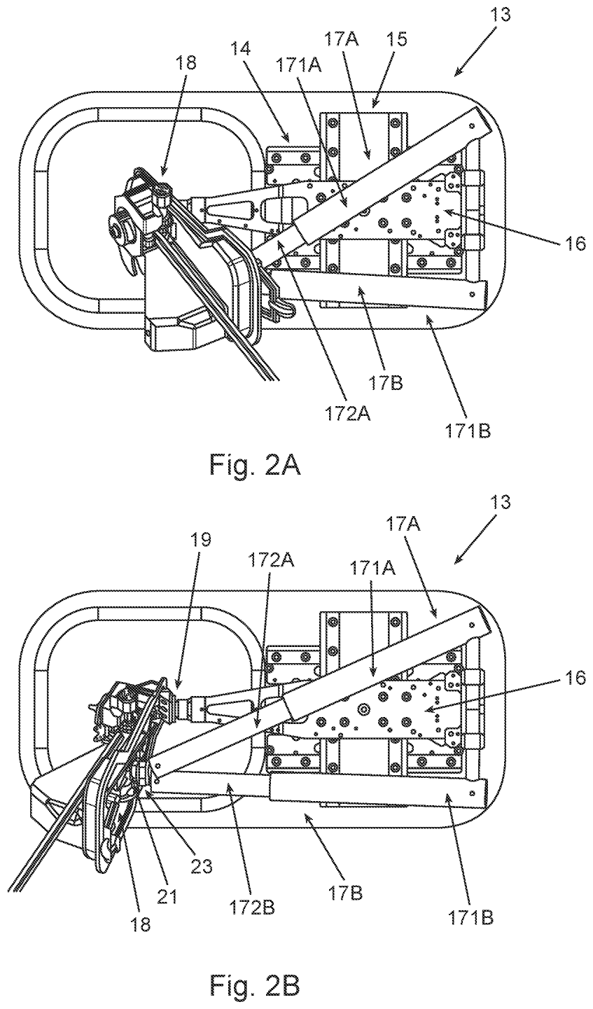

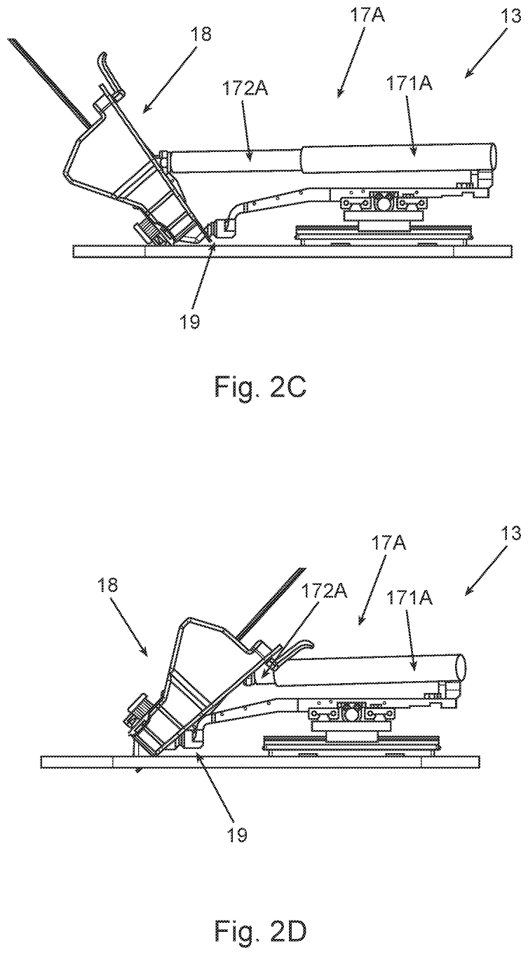

Reference is now made to FIGS. 2A-2D, which show schematic top (FIGS. 2A-2B) and side (FIGS. 2C-2D) views of exemplary optional movements of the steering mechanism of the medical device, according to some embodiments. According to some exemplary embodiments, the device 13 may include a stationary platform 14, a first moveable platform 15, a second moveable platform 16, two piston mechanisms 17A and 17B coupled to the second moveable platform 16, each piston mechanism including a cylinder 171A and 171B, a piston 172A and 172B, at least a portion of which is positioned within the cylinder, and a driving mechanism (not shown in FIGS. 2A-2D) configured to controllably propel the piston in and out of the cylinder. The device 13 may further include an end effector 18 which includes, or to which is coupled, at least a portion of the insertion mechanism, which is configured to impart longitudinal movement to the medical instrument. In some embodiments, the lower portion of the end effector 18 may be coupled to a lower gimbal 19, which is connected to the second moveable platform 16, and the top portion of the end effector 18 may be connected to a top gimbal 21, which is connected to the distal ends of the pistons 172A and 172B. In some embodiments, the distal ends of the pistons 172A and 172B may be coupled to a common joint 23, which in turn is coupled to the top gimbal 21. In some embodiments, the cylinders, pistons and the common joint may all be located substantially in a single plane, allowing larger angular movement and thus a larger workspace for the device's control head 18 and medical instrument, as described, for example, in abovementioned U.S. Patent Application Publication No. 2019/290,372.

In some embodiments, the medical device 13 may have a plurality of degrees of freedom (DOF) in operating and controlling the movement of the medical instrument along one or more axis. For example, the device may have up to six degrees of freedom. For example, the device may have at least four degrees of freedom. For example, the device 13 may have five degrees of freedom, including: forward-backward linear translation facilitated by the stationary platform 14 and a first moveable platform 15 which can move on top of the stationary platform 14, left-right linear translation facilitated by the first moveable platform 15 and a second moveable platform 16 which can move on top of the first moveable platform 15, front-back and left-right rotations facilitated by two piston mechanism 17A and 17B connected to the second moveable platform 16, and longitudinal translation toward the subject's body facilitated by the device's insertion mechanism (not shown), as disclosed in abovementioned U.S. Patent Application Publication No. 2019/290,372. In some embodiments, the device may have six degrees of freedom, which include the five degrees of freedom described above and, in addition, rotation of the medical instrument about its longitudinal axis.

FIG. 2A illustrates a top view of a state in which the first moveable platform 15 and the second moveable platform 16 are in a “neutral” position, i.e., the first moveable platform 15 is positioned at the center of the stationary platform 14 and the second moveable platform is positioned at the center of the first moveable platform 15. Further illustrated in FIG. 2A are the two piston mechanisms 17A and 17B, whereby the piston 172A of one of the piston mechanisms 17A is fully extended out of its cylinder 171A while the piston of the second piston mechanism 17B is positioned within its cylinder 171B (thus not being visible in FIG. 2A). At this state, the control head 18, being coupled at its lower end to the lower gimbal 19 and at its top end to the piston mechanisms via the top gimbal 21 and the common joint 23 (shown in FIG. 2B), rotates to the left (i.e., toward the piston mechanism 17B) and to the back (i.e., to the distal end of the device). FIG. 2B illustrates a top view of a state similar to that illustrated in FIG. 2A, except that in FIG. 2B piston 172B is partially extended out of its cylinder 171B, resulting in forward rotation of the end effector 18 about its lower end. FIG. 2C illustrates a side view of a state in which the first moveable platform 15 and the second moveable platform 16 are in a “neutral” position, as shown in FIGS. 2A-2B, and both pistons 172A and 172B of the two piston mechanisms 17A and 17B, respectively, are fully extended out of their respective cylinders 171A and 171B. At this state, the control head 18, being coupled at its lower end to the lower gimbal 19 and at its top end to the piston mechanisms via the top gimbal 21 and the common joint 23, rotates forward about its lower end, which is coupled to the lower gimbal 19, while being maintained substantially at the center of the device. FIG. 2D illustrates a top view of a state similar to that illustrated in FIG. 2C, except that in FIG. 2D both pistons 172A and 172B are fully drawn back into their respective cylinders 171A and 171B, resulting in backward rotation of the end effector 18 about its lower end.

It can be appreciated, that “mirror” states of those illustrated in FIGS. 2A-2B can be achieved by fully extending the piston 172B out of its cylinder 171B, and propelling piston 172A in and out of its cylinder 171A. Further, numerous additional states can be achieved by simultaneously translating the first moveable platform 15 along the stationary platform 14 and, separately or simultaneously, translating the second moveable platform 16 along the first stationary platform 15.

As exemplified in FIGS. 2A-2D, the steering mechanism of the medical device 13 is capable of performing a large range of movements in various directions and various angles. Hence, a dedicated cover, that can cover and shield the inner elements of the device should be configured such that is does not compromise the range of movements of the steering mechanism, so as not to affect its functioning.

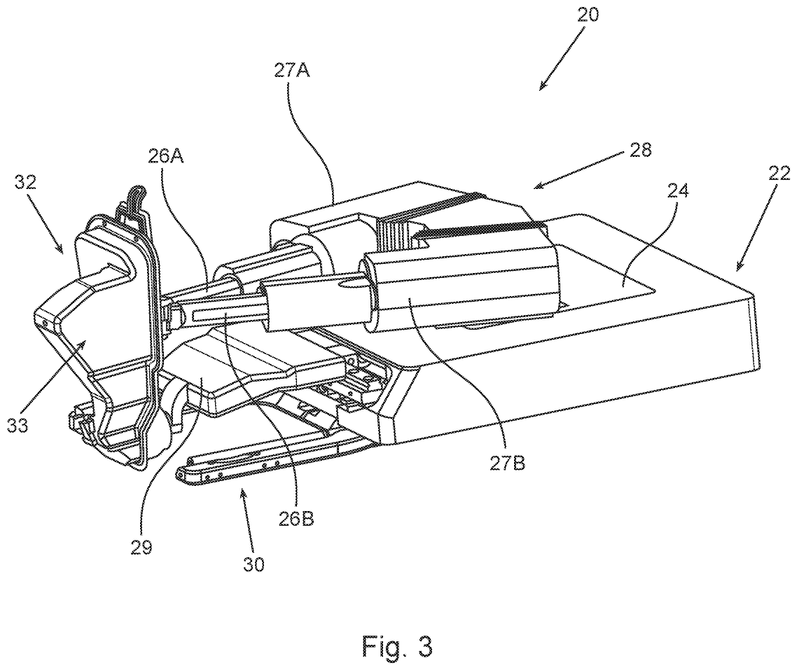

Reference is now made to FIG. 3, which shows a schematic illustration of a medical device for inserting and steering a medical tool, having an adjustable protective cover, according to some embodiments. The protective cover may include a plurality of individual cover elements/housings, some of which may be associated with one another. As shown in FIG. 3, the protective cover may include a housing 22 accommodating therein at least a portion of the steering mechanism. The steering mechanism may include at least one moveable platform (not shown) and at least two moveable arms 26A and 26B, configured to allow or control movement of an end effector (“control head”) 32. As detailed above, the moveable arms may be configured as piston mechanisms. To the end of the control head 32, a suitable medical instrument (not shown) may be connected. Additionally, as shown in FIG. 3, a protecting cover 33 may be placed on/attached to the end effector 32. Further shown is base 30, which may be configured to allow placing the medical device on or in close proximity to a body of a subject. Also shown in FIG. 3 are cover elements 27A-27B and 28, configured to at least partially cover and protect the moveable arms 26A and 26B and the operating elements associated therewith.

Additionally, covering element 29, may be used to cover/encase an extending arm which may be coupled to the end effector, e.g., via a gimbal, and configured to affect movement thereof. In some embodiments, the extending arm is an extension of a moveable platform of the steering mechanism. Further shown is a top adjustable cover 24, which is configured to allow sealing/covering of the inner portions of the steering mechanism, while allowing the operation of the elements thereof, i.e., maintaining coverage and protection of the inner portion of the device, while allowing movement of the various elements of the steering mechanism, by being moved therewith.

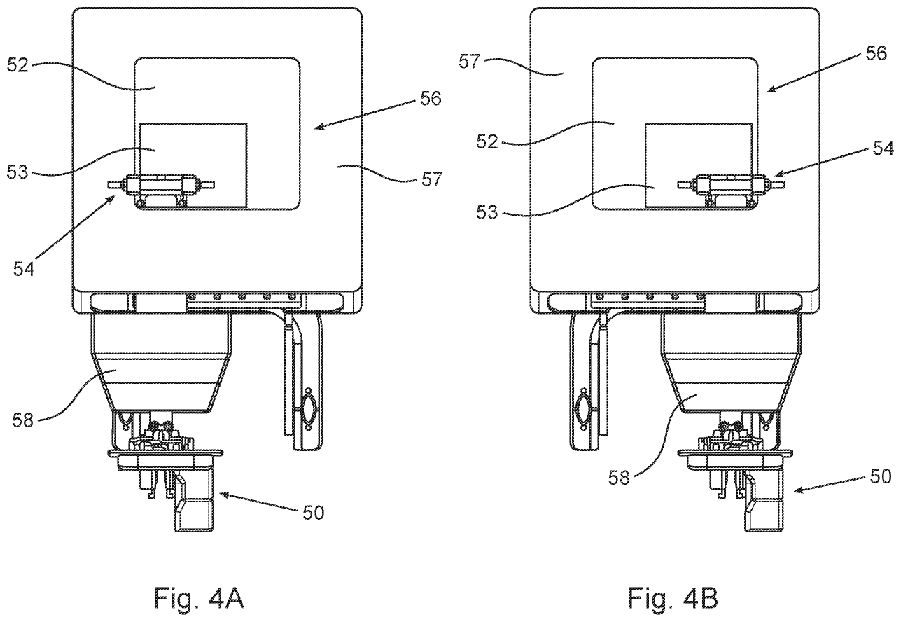

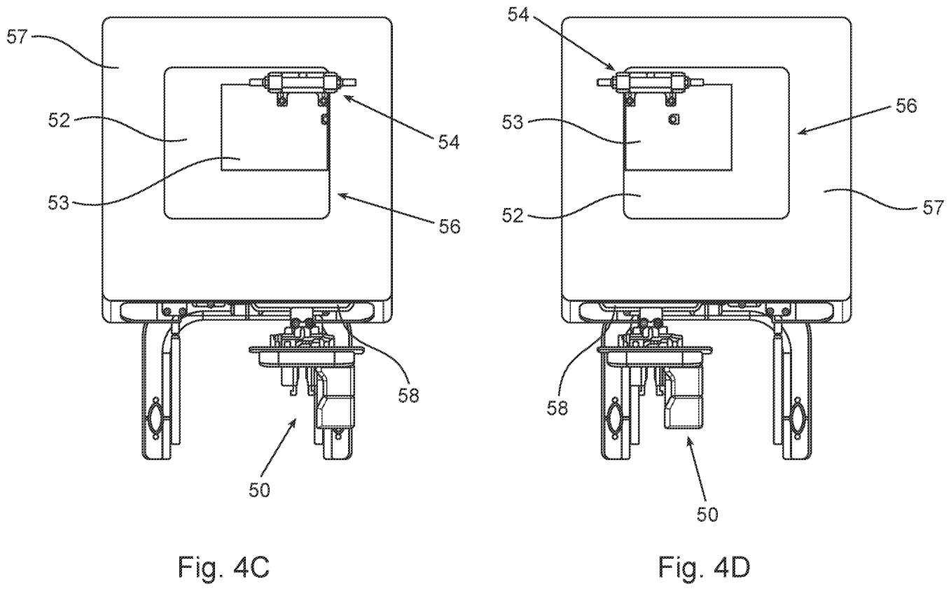

Reference is now made to FIGS. 4A-4D which show top views of the medical device shown in FIG. 3, illustrating the adjustable cover 24 at various positions, according to some embodiments. For simplicity, the elements of the top portion of the steering mechanism (in particular, the moving arms and the elements associated therewith (such as pistons, cylinders, motors, axels, cover elements, etc.)) are omitted from FIGS. 4A-4D. Shown in FIGS. 4A-4D are end effector 50 and extending arm 58 associated therewith, which allows the movement thereof (for example, in forward-backward direction). In some embodiments, the extending arm 58 is an extension of a top moveable platform of the steering mechanism. Also shown is a top base 54, which is used, inter alia, to couple the top portion of the steering mechanism (including the moving arms and the elements associated therewith) to the inner parts thereof (e.g., to the top moveable platform). Further shown is adjustable cover 56, which includes an inner moveable cover portion 53 and an outer moveable cover portion 52. The moveable cover portions 52 and 53 are configured to change their relative locations, according to the movement of the steering mechanism, while maintaining coverage integrity of the inner components of the device. The adjustable cover 56 may be rigid or semi rigid. The inner cover portion 53 may be attached or associated with the top moveable platform, and the outer cover portion 52 may be a “floating” cover, associated with a static casing 57. FIGS. 4A-4D show extending arm 58 and the end effector 50 coupled thereto at various positions, and the corresponding positions of the moveable cover portions 52 and 53, in all of which, the adjustable cover 56 maintains coverage of the inner components of the steering mechanism of the device, while allowing full range of movement of the end effector. In FIG. 4A the extending arm 58 and the end effector 50 are extended outward and are at their most right-hand position, the corresponding position of the inner cover portion 53 being at/near the front right-hand corner of an opening formed in the top region of the static casing 57. In FIG. 4B the extending arm 58 and the end effector 50 are extended outward and are at their most left-hand position, the corresponding position of the inner cover portion 53 being at/near the front left-hand corner of the opening of the static casing 57. In FIG. 4C the extending arm 58 and the end effector 50 are retracted inward and are at their most left-hand position, the corresponding position of the inner cover portion 53 being at/near the back left-hand corner of the opening of the static casing 57. In FIG. 4D the extending arm 58 and the end effector 50 are retracted inward and are at their most right-hand position, the corresponding position of the inner cover portion 53 being at/near the back right-hand corner of the opening of the static casing 57.

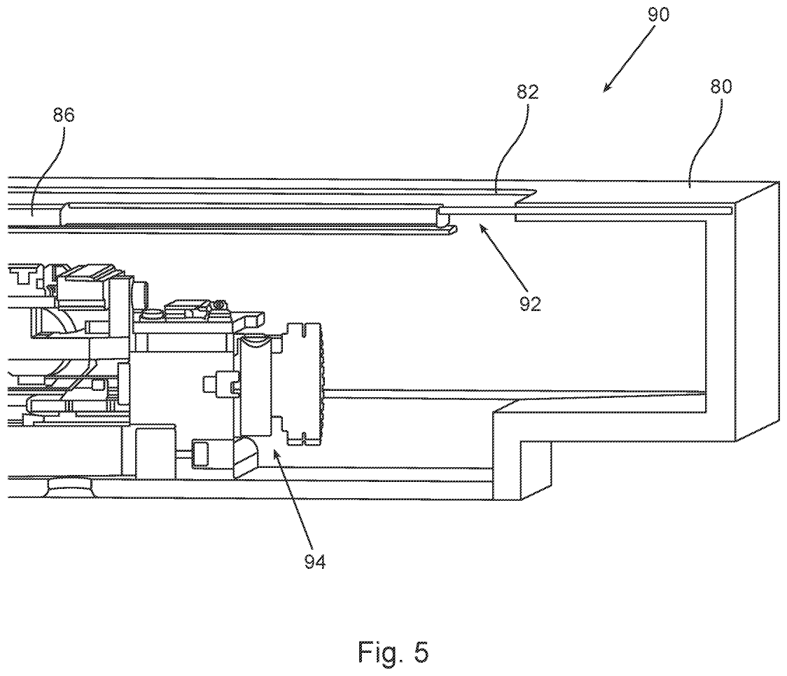

Reference is now made to FIG. 5, which shows a cross-section of the external housing and an adjustable top protective cover of a medical device (such as that shown in FIGS. 3 and 4A-4D), according to some embodiments. As shown in FIG. 5, housing 90 includes an external static casing 80, partially covering the inner parts/components 94 of the medical device. The external casing 80 includes an opening 92 on a top region thereof. The opening 92 is covered by an adjustable cover which may include several portions (elements), to allow the adjustable covering of the opening 92 of the static casing 80, while maintaining full range of movement of the components of the steering mechanism. The adjustable cover includes at least an inner cover portion 86 (also referred to as “inner moveable cover portion” in FIGS. 4A-4D), which is attached to the top moveable platform (not shown) of the steering mechanism (the extending arm optionally being an extension thereof), and an outer cover portion 82 (also referred to as “outer moveable cover portion” in FIGS. 4A-4D), which may be a floating cover that can be moved to a desired location by the top moveable platform of the steering mechanism. The adjustable cover may further include a sealing layer 84 positioned between the outer and inner cover portions, which can be a sealing element (such as a Teflon sealing element), that can slide or move over the inner cover 86. Thus, the set of cover portions (elements) can act as moving “shades” or “shutters”, whereby the various cover portions can slide/move one over the other, according to the movement of the steering mechanism, while maintaining the coverage of the top opening 92 of the external casing 80 and while allowing full movement of the steering mechanism.

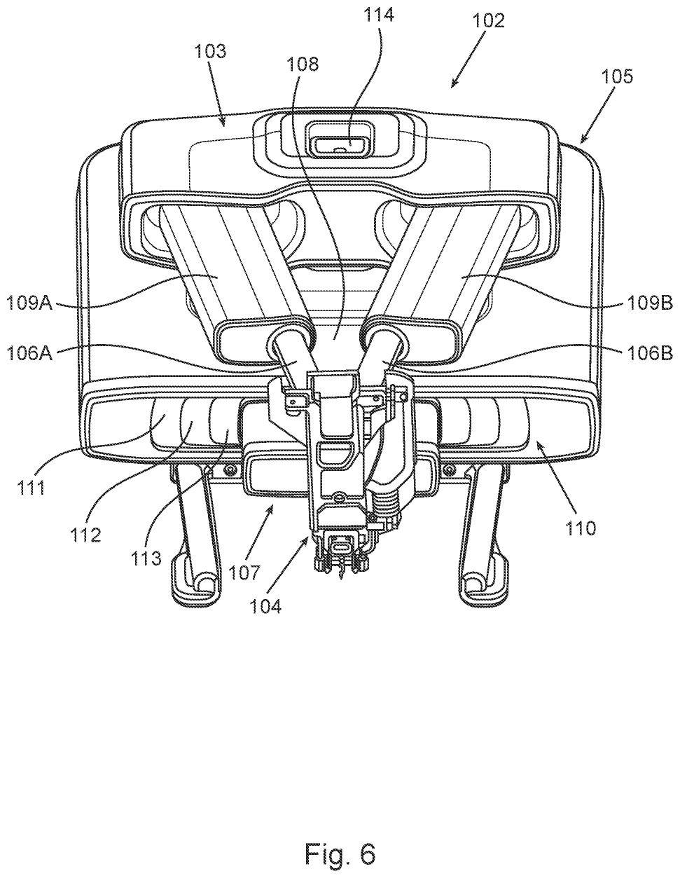

Reference is now made to FIG. 6, which illustrates a schematic view of a medical device, having an adjustable cover, according to some embodiments. As shown in FIG. 6, the medical device 102 may be covered by an external casing 105 and an associated adjustable cover 108, which allows the uncompromised movement of elements of the steering mechanism of the device (including, for example, moving arms 106A and 106B and an extending arm 107 (shown covered) and consequently of the end effector 104, while covering the top opening of the casing 105. In some embodiments, the moveable arms 106A and 106B may be covered, at least in part, by separate arm covers 109A and 109B, respectively, and an additional top cover 103 may be used to cover the proximal ends of the moveable arms and the actuators/motors (not shown) which control the operation of the moveable arms. In some embodiments, a button 114 may be disposed on one of the cover elements, for example on top cover 103, as shown in FIG. 6, to allow the user to manually activate the steering mechanism and/or the insertion mechanism. In some embodiments, the external casing 105 may further include a front opening substantially perpendicular to the top opening, through which the extending arm may extend. A second adjustable cover 110 may be employed to cover the front opening. The front adjustable cover 110 may include front moveable cover elements, such as front moveable cover elements 111, 112 and 113, which may surround the extending arm (or the cover thereof) and at least partially overlap with each other, such that left-right movement of the extending arm moves the front cover portions 111, 112 and 113, while maintaining coverage of the front opening of the casing 105.

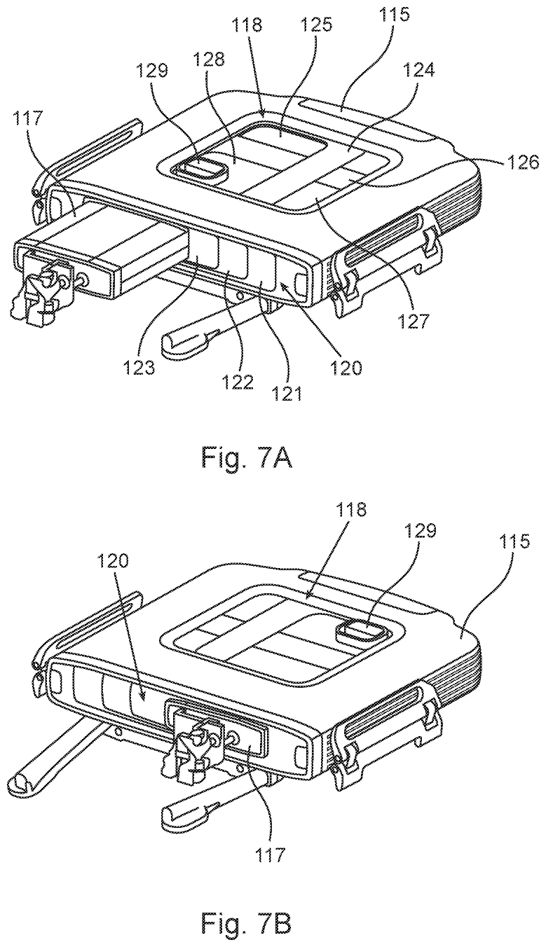

Reference is now made to FIGS. 7A-7B, which illustrate a perspective view of the medical device of FIG. 6 and exemplary states of the adjustable protective covers of the medical device, according to some embodiments. For simplicity, the elements of the top portion of the steering mechanism (in particular, the moving arms and the elements associated therewith) and the device's control head are omitted from FIGS. 7A-7B. As shown in FIGS. 7A-7B, the device may include a top adjustable cover 118 and a front adjustable cover 120. The front adjustable cover 120 may be substantially perpendicular to the top adjustable cover 118. The top adjustable cover 118 may include a plurality (e.g., two, three, four, etc.) of partially overlapping moveable cover elements. For example, as shown in FIGS. 7A-7B, the top adjustable cover 118 may include four moveable cover elements 124, 125, 126 and 127. The moveable cover elements may be configured as a substantially flat frame. A complimentary cover element 128 may be attached to a base 129, which couples the top portion of the steering mechanism (including the moving arms and the elements associated therewith) to the inner parts thereof (e.g., to a top moveable platform which includes the extending arm). The front adjustable cover 120 may also include a plurality (e.g., two, three, four, etc.) of partially overlapping moveable cover elements. For example, as shown in FIGS. 7A-7B, the front adjustable cover 120 may include three moveable cover elements 121, 122 and 123. Further shown is extending arm 117 (shown covered), which can protrude outward or retract inward through the front opening, as well as move sideways within the front opening. In FIG. 7A, the extending arm 117 is shown protruding outward and moved sideways to a right-hand side position, and in FIG. 7B, the extending arm 117 is shown retracted inward (into the external casing 115), and moved sideways to a left-hand side position, with the moveable cover elements 121, 122 and 123 of the front adjustable cover 120 moving (sideways) therewith, such that the front opening of the external casing 115 is maintained covered. The adjustable covers, by the array of covering elements thereof, enable maintaining closing of the top and/or front opening during movement of the elements of the steering mechanism, for example movement of the extending arm 117, in any desired direction (for example, left-right/forward-backward). The array of covering elements act as at least partially overlapping shades (or “shutters”) that move relative to each other and relative to the steering elements of the medical device. In some embodiments, the front adjustable cover 120 and the top adjustable cover are configured to operate simultaneously. For example, left-right movement of steering elements of the medical device sideways, may cause simultaneous movement of cover elements of both the top and front adjustable covers. Specifically, as shown in FIGS. 7A-7B, left-right movement of the top moveable platform (represented in FIGS. 7A-7B by the base 129 and the extending arm 117) causes simultaneous movement of the cover elements of the top and front adjustable covers, which enables full coverage of the top and front openings of the external casing 115.

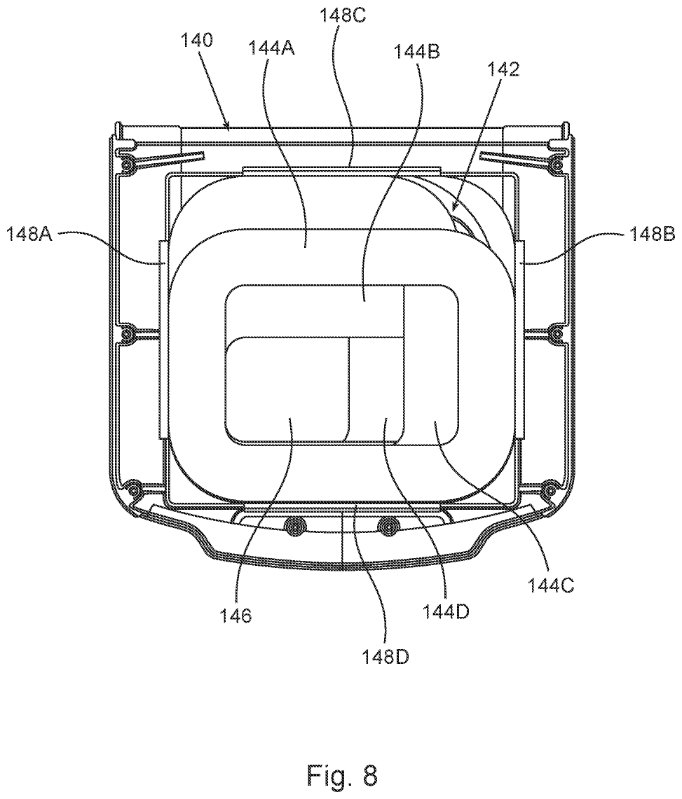

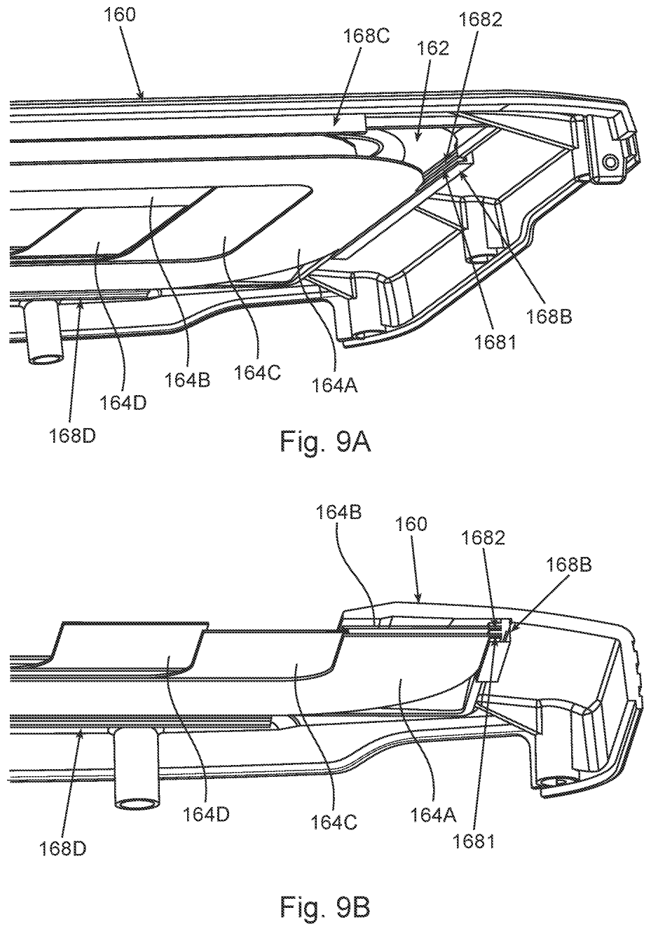

Reference is now made to FIG. 8, which illustrates a bottom view of a top portion of the external casing and the top adjustable protective cover shown in FIGS. 7A-7B, according to some embodiments. As shown in FIG. 8, the adjustable cover 142 includes moveable cover elements (shown as elements 144A-144D). Further shown is an opening 146 in the adjustable cover 142. Opening 146 may be covered by a complimentary cover element (not shown in FIG. 8), which may be attached to a base coupling the top portion of the steering mechanism to the inner parts thereof. In some embodiments, a plurality of guides may be disposed on the bottom (inner) surface of the top portion of the external casing 140, to support and/or guide the movement of the top cover elements 144A-144D. The number of guides may correspond to the number of cover elements or, in some embodiments, guides may not be required for all the cover elements, and therefore, there may be fewer guides than cover elements. In some embodiments, each cover element which is supported by guides, may be supported by two guides disposed opposite each other, such that the two guides support two opposite ends of the cover element. As shown in FIG. 8, four guides 148A-148D may be disposed on four sides of a rectangle (e.g., a square) shape formed on the inner surface of the top portion of the casing 140. In some embodiments, the cover elements may be “divided” into pairs, such that each two cover elements are supported by the same two guides disposed on opposite sides of the rectangle. For example, the ends of moveable cover elements 144A and 144B (two opposite ends of each of the two cover elements) may be supported by guides 148A and 148B and the ends of moveable cover elements 144C and 144D (two opposite ends of each of the two cover elements) may be supported by guides 148C and 148D. In some embodiments, each guide may include slots/rails formed therein, such that each rail is used to guide/support one cover element (as shown in FIGS. 9A-9B). For example, as shown in FIG. 9B, each guide may include two rails, to support two cover elements. The moveable cover elements may be identical, similar or different in size, shape, composition, thickness, and the like. In some embodiments, the moveable cover elements are similar in size, shape and thickness. In some embodiments, the moveable cover elements at least partially overlap. In some embodiments, the moveable cover elements may be located in different planes and may be slidable/movable relative to each other and/or relative to the external casing 140 of the device. In some embodiments, at least some of the moveable cover elements may overlap. In some embodiments, at least some of the cover elements may at least partially overlap. In some embodiments, the top cover elements 144A-144D act as slidable shutters/shades/blinds. In some embodiments, the top cover elements at least partially overlap along two sides/faces thereof. In some embodiments, the moveable cover elements may be made of metal, plastic, aluminum, and/or the like. In some embodiments, the cover elements are movable by movement of the steering mechanism of the device, without bending of the elements, and with smooth movement thereof, which does not compromise or otherwise affect the movement of the elements of the steering mechanism. The durability of the cover elements and the minimal to no friction between the cover elements and the guides and/or between the cover elements themselves may be enabled or facilitated by the materials and/or coating of the cover elements and the guides. In some embodiments, the adjustable cover elements are movable or slidable one over the other. According to some embodiments, the thickness of the top adjustable cover (i.e., the thickness of the moveable cover elements stacked together) may be in the range of about 1-10 mm. In some embodiments, the thickness of the adjustable cover may be in the range of about 2-6 mm. In some embodiments, the thickness of the adjustable cover may be in the range of about 3-5 mm. According to some embodiments, the thickness of the adjustable cover may be in the range of about 3.5-4.5 mm. According to some embodiments, the thickness of the adjustable cover may be in the range of about 3.75-4.25 mm. Advantageously, arranging the cover elements and the corresponding guiding rails in a crosshatch pattern (as shown in FIG. 9B below, for example), assists in maintaining a relatively low thickness of the adjustable cover. In some embodiments, the size of the adjustable cover may be in the range of about 100-200 mm. In some embodiments, the range of movement (travel distance) of the adjustable cover may be in the range of about 50-100 mm. In some embodiments, the ratio between the travel of the adjustable cover and its size is in the range of about 40-65%. In some exemplary embodiments, the ratio between the travel of the adjustable cover and its size is about 47%. In some embodiments, the ratio between the thickness of the adjustable cover and its travel is in the range of about 5-15%. In some exemplary embodiments, the ratio between the thickness of the adjustable cover and its travel is about 10%. The ratio between the travel and the size of the adjustable cover and the ratio between the thickness of the cover and its travel may be mutually dependent, such that the ratio between the travel and the size of the adjustable cover can be increased by increasing the ratio between the thickness and the travel. According to some embodiments, the cover elements are sliding shutters, configured to slide one against the other, while maintaining their structure (i.e., without bending or otherwise deforming), despite their relatively low thickness. In some embodiments, one or more bearings (not shown), such as ball bearings, may be used in order to facilitate the movement of the cover elements (e.g., shutters). The bearings may be used in the plane of motion and/or in the plane perpendicular to the plane of motion, and they may be positioned on the casing and/or on one or more of the cover elements.

Reference is now made to FIGS. 9A-9B, which show perspective bottom views of the top portion of the external casing and the adjustable protective cover shown in FIG. 8, according to some embodiments. As shown in FIG. 9A, which illustrates a bottom view (i.e. the side that faces the inner region of a medical device), the adjustable cover 162 includes several adjustable cover elements (shown as elements 164A-164D), which are shown as sliding shutters, each located on a different plane, such that the elements can slide one over the other in a smooth and effective manner, while maintaining overlap therebetween. As shown in FIG. 9A, guides 168B-168D (the fourth guide is not shown in FIG. 9A) include guiding rails (for example, rails 1681 and 1682 formed in guide 168B) within which the edges of the cover elements can slide/move in a desired direction, as dictated by the movement of the corresponding elements of the steering mechanism. FIG. 9B is a cross-sectional perspective bottom view of the adjustable cover shown in FIG. 9A. According so some embodiments, as shown in FIG. 9B, the edges of both cover elements 164A and 164B are supported within rails 1681 and 1682, respectively, formed in guide 168B. Similarly, the opposite edges of cover elements 164A and 164B may be supported within rails formed in a guide (not shown) located opposite guide 168B, and the edges of cover elements 164C and 164D may be supported within rails formed in guides 168C and 168D, which are located opposite each other. In some embodiments, the rails in all the guides are arranged in a crosshatch pattern, so as to arrange the cover elements they support in a crosshatch pattern. The cover elements are slidable/movable one over the other and may at least partially overlap along two faces of each. The relative location of the adjustable cover elements allows movement in various linear directions, including left-right and/or inward-outward. By the relative and smooth movement of the cover elements, whereby each set of elements is located on a different height relative to the other, full coverage of the opening is achieved, while maintaining full movement range of the respective elements of the steering mechanism.

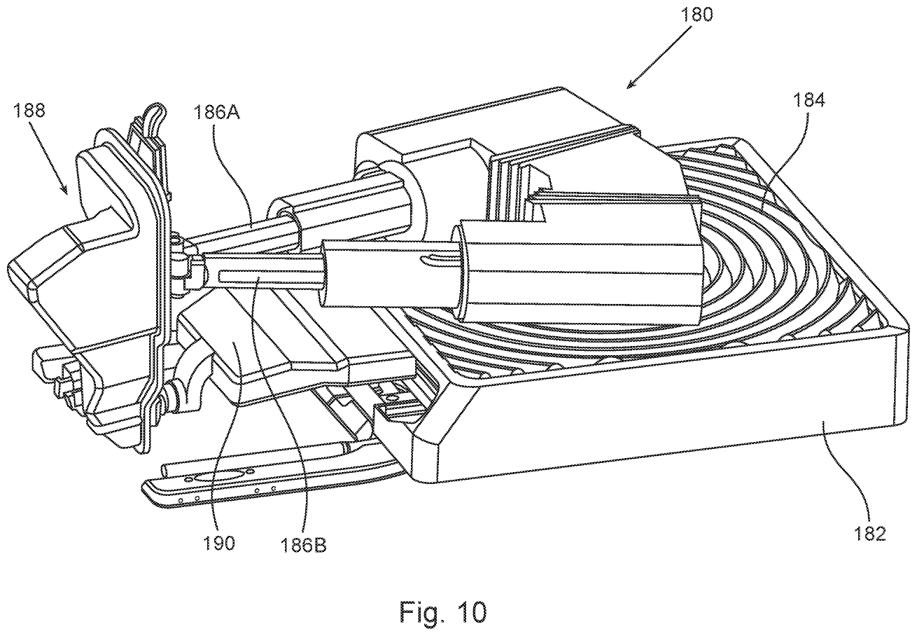

Reference is now made to FIG. 10, which illustrates a medical device having a flexible top protective cover, according to some embodiments. As shown in FIG. 10, device 180 includes a rigid housing 182 and a flexible protecting cover 184. Further shown is a top portion of the steering mechanism of the device, which includes, inter alia, movable arms 186A-186B and extending arm 190, configured to maneuver the end effector 188, e.g., forward-backward movements thereof. Protective cover 184, is shown in the form of a flexible top cover having ridges, which cover the top portion of the device, thereby protecting the inner components (not shown) within the rigid housing 182 of the device, without compromising the movement and maneuverability of the steering mechanism (in particular the top portion thereof). Due to the flexibility of the adjustable top cover, the movement of the top portion of the steering mechanism is not compromised.

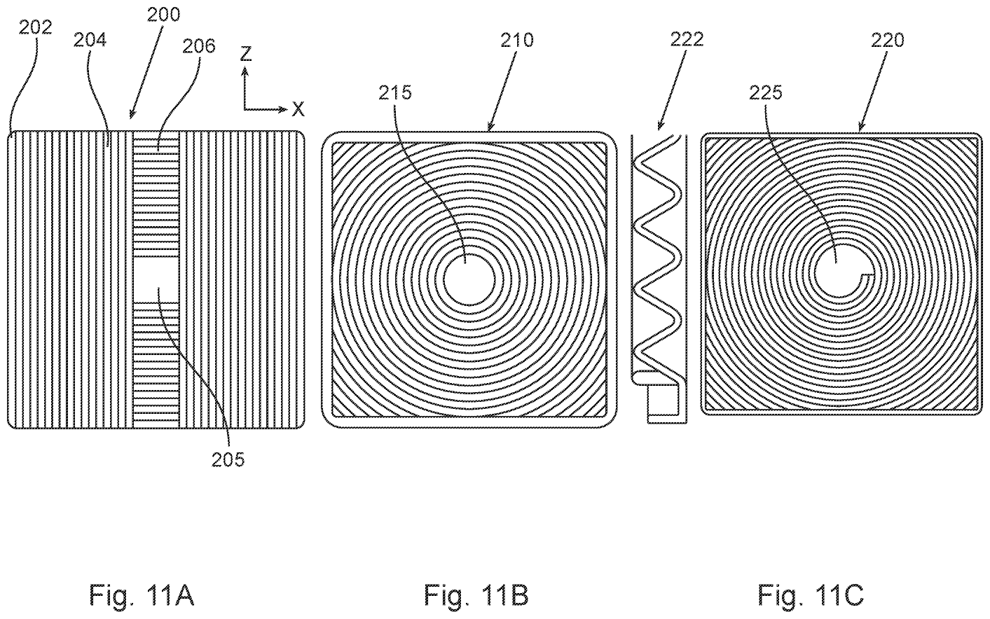

Reference is now made to FIGS. 11A-11C, which illustrate schematic top views of various flexible top protective covers, according to some embodiments. As shown in FIG. 11A, the protective cover 200 may be made of flexible material having straight lines of ridges, which are arranged in layers, forming a center opening 205, through which the base of the top steering mechanism element/s can protrude from the inner compartment of the device. The outer circumference/side 202 of the flexible cover may be fixed to the rigid housing of the device. Flexible layer 204 may be free to move over the Z axis (relative to the top (piston) platform of the top portion of the steering mechanism). Flexible layer 206 may be free to move over the X axis. Shown in FIG. 11B is another example of a flexible adjustable top protective cover 210, which is in a form of round ridges, having a center opening 215, for the base of the top elements of the steering mechanisms. Due to its flexibility and round shape, the adjustable top cover 210 allows free movement of the top elements of the steering mechanism in various directions, while maintaining full coverage of the top of the housing of the device. Shown in FIG. 11C is another example of a flexible adjustable top protective cover 220, which is in a form of a spiral ridges (shown enlarged as ridges 222), having a center opening 225, for the top elements of the steering mechanisms. Due to its flexibility and spiral shape, the adjustable top cover 220 allows free movement of the top elements of the steering mechanism in various directions, while maintaining full coverage of the top of the housing of the device. In some embodiments, the flexible top protective cover may be in the form of a sheet of soft material and rigid ribs/ridges, which may be spiral or discrete, having constant or differential widths.

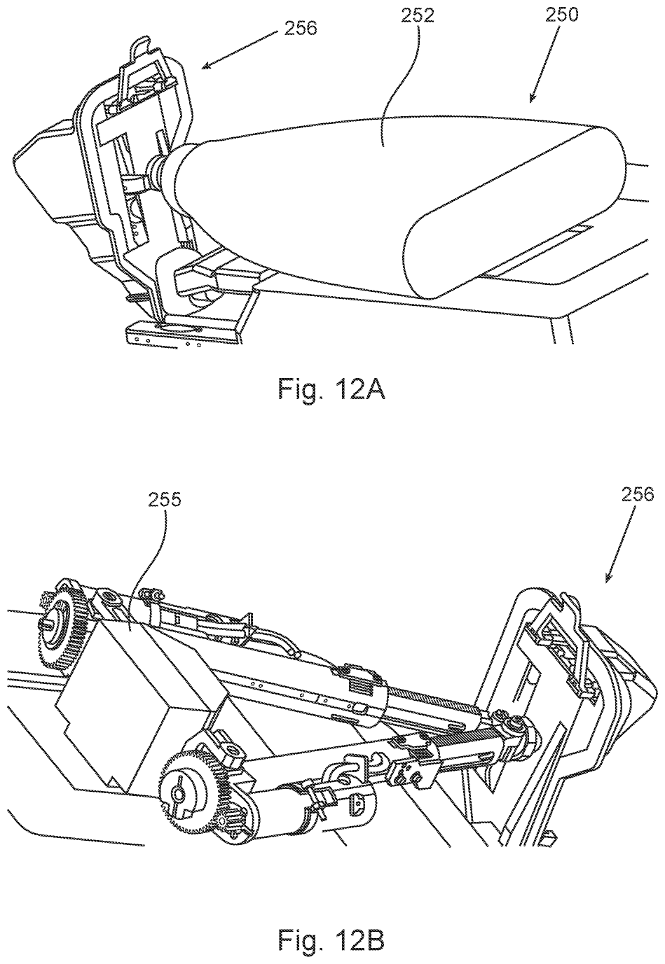

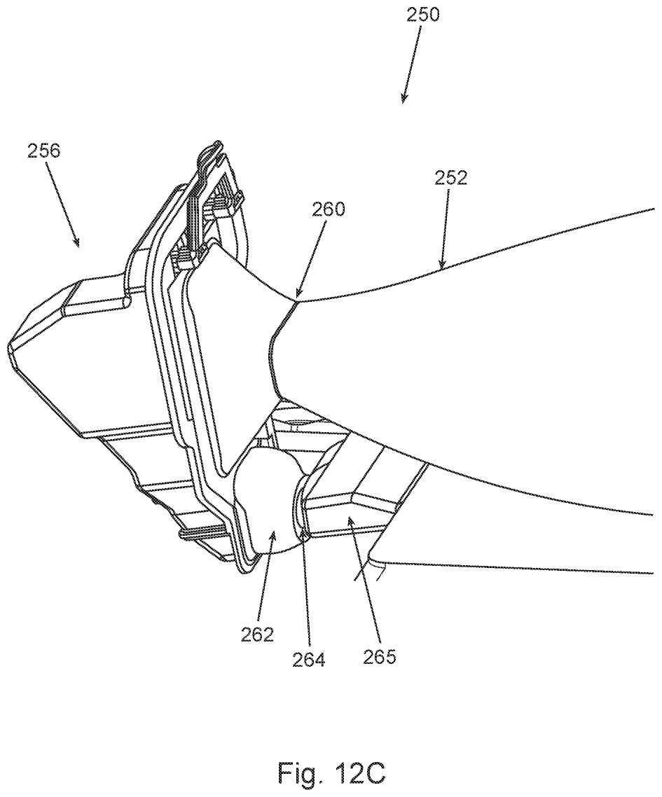

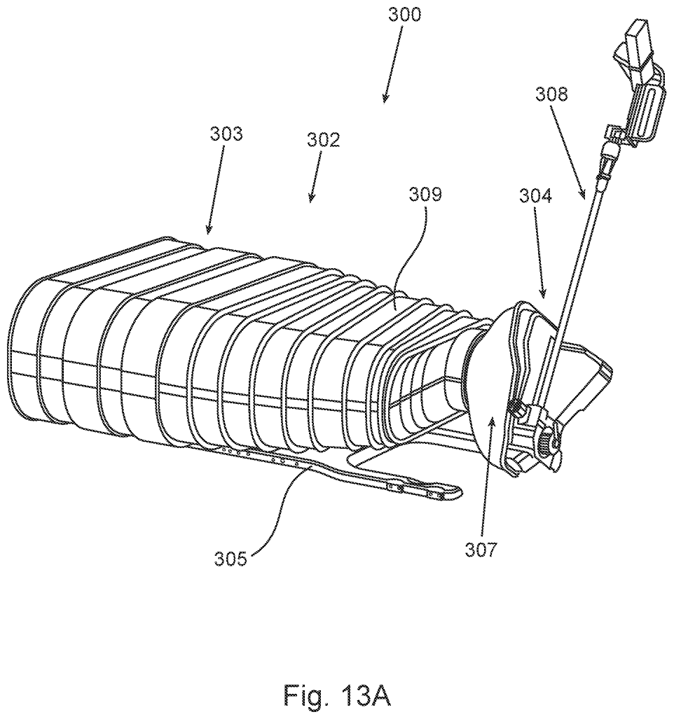

Reference is now made to FIGS. 12A-12C, which illustrate schematic views of flexible adjustable protective covers of elements of the steering mechanism of a medical device, according to some embodiments. As shown in FIG. 12A, the top elements of steering mechanism of device 250 (e.g., including two moveable arms) may be covered with an adjustable cover 252, which may at least partially cover the steering elements (such as the moving arms) and protect them from environmental deposits, such as, dust and/or bodily fluids from a subject. The adjustable cover may be made of any suitable material, such as, rubber, silicone, plastic, and the like. In some embodiments, the adjustable cover 252 is soft. In some embodiments, the adjustable cover 252 is flexible. In some embodiments, the adjustable cover 252 is configured to cover the entire area of the top elements of the steering mechanism, including, inter alia, the moveable arms, motors, etc., up to their interaction region with the end effector 256. In some embodiments, the adjustable cover 252 is configured to cover only the moveable arms. Shown in FIG. 12B, is inner rigid cover 255, which may be used to cover portions of the top elements of the steering mechanism. The inner rigid cover 255 may be made of any suitable material, such as, plastic and/or metal. The inner cover 255 may be configured to cover and protect electronic components of the steering mechanism utilized for controlling the operation of the steering mechanism, as well as any additional components, including, motors, shaft, brushes, hinges, and the like. In some embodiments, the inner cover 255 may be used in addition to the adjustable cover 252 and covered therein. In some embodiments, the adjustable cover 252 may cover only the moveable arms, and it may be attached to the rigid cover 255, which covers the electronic components (e.g., motors) of the steering mechanism. Reference is now made to FIG. 12C, which shows a perspective view of the end effector 256 region of a medical device 250. In some embodiments, as shown in FIG. 12C, the adjustable cover 252 may be configured to cover the entire area of the top elements of the steering mechanism, including, inter alia, the moveable arm, pistons, motors, etc., up to their interaction region with the end effector 256. The interaction region at the common joint and a top gimbal (not shown) may be continuously covered with cover 252, or optionally, covered by a separate discrete cover. At the connection region with the end effector, a slip bearing 260 may be placed, to allow the cover of the common joint and top gimbal to rotate relatively to the adjustable cover 252. Further shown in FIG. 12C, is an additional flexible cover element 262, which may be used to cover the lower hinge/gimbal which connects between the lower extending arm 265 and the end effector. Additionally, a rigid ring 264 may be mounted at the end of the cover 262, to allow free movement of the lower gimbal.

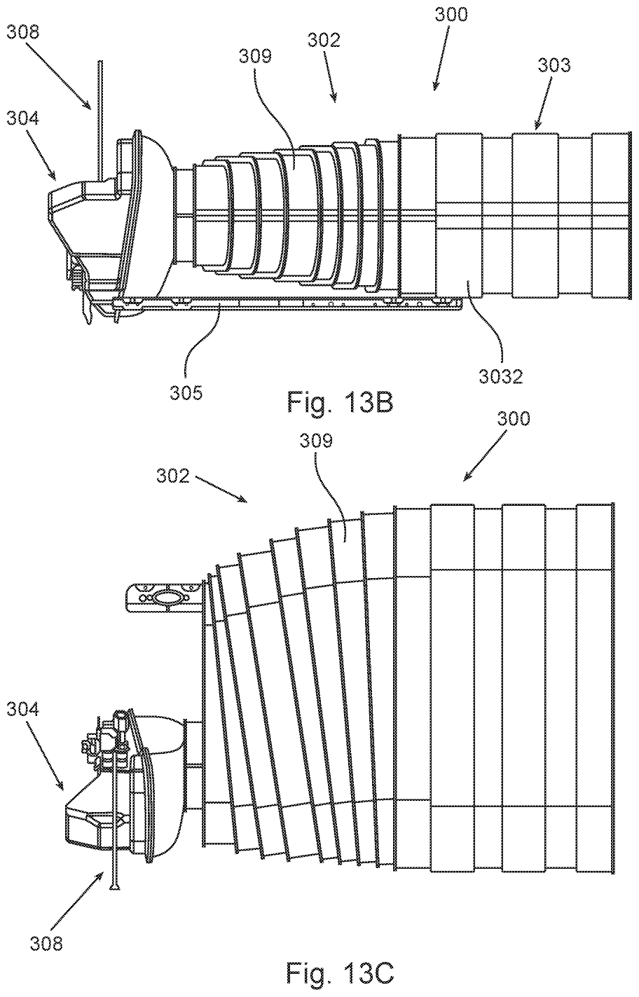

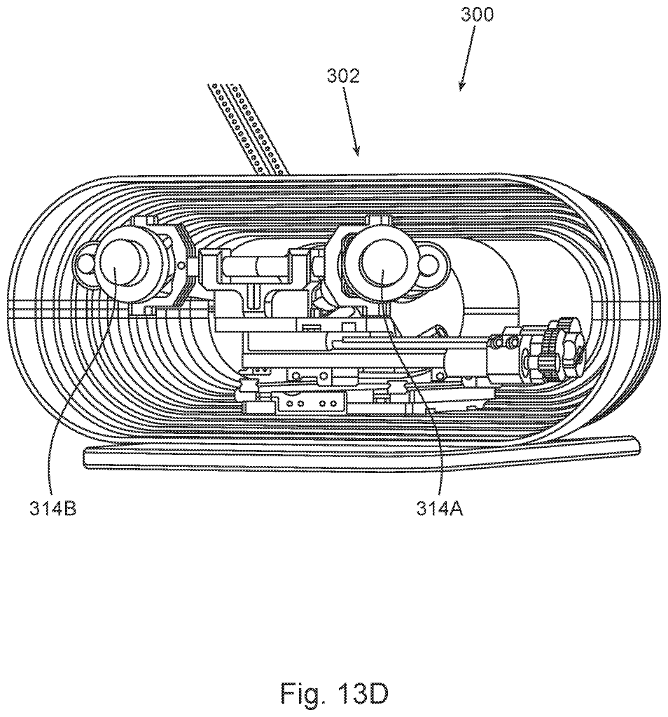

Reference is now made to FIGS. 13A-13D, which illustrate schematic views of an adjustable protective cover covering elements of the steering mechanism of a medical device, according to some embodiments. Shown in FIG. 13A is a perspective view of an exemplary medical device 300, having an end effector 304, configured to insert a medical tool 308 into a body of a subject. The steering mechanism (not shown) of the device is covered by an adjustable protective cover 302. Adjustable protective cover 302 shown in FIG. 13A is a collapsible/retractable (e.g., telescopic, scale-like) cover, configured to extend/open or collapse/close, as well as move and/or rotate/curve sideways, in accordance with the movement of the steering mechanism. In some embodiments, the protective cover 302 may have a straight tube-like shape, when the steering mechanism is in its neutral position. The protective cover 302 may be made of flexible, semi-flexible or rigid material. In some embodiments, at least part of the protective cover 302 may be constructed from partially overlapping consecutive elements 309. The cover elements 309 may have an annular shape. In some embodiments, each element may have a smaller size relative to the previous element, such that each element may be at least partially inserted within the previous element. In some embodiments, movement of the steering mechanism of the medical device may cause one or more of the cover elements 309 to be further inserted into the previous element or moved partially out of the previous element. In some embodiments, a flexible and/or sealing material may be disposed between adjacent elements of the cover. In some embodiments, the rear (proximal) portion 303 of the adjustable cover (i.e., the portion that is farthest from the end effector) is attached/mounted/fixed to the base 305 of the medical device, and it may be stationary, such that it does not move relative to the base of the device during operation of the device (e.g., during movement of the steering mechanism). In some embodiments, the rear portion 303 of the adjustable cover is the largest element of the adjustable cover. In some embodiments, a front (distal) portion of the cover may cover the top and/or bottom gimbals connecting the end effector to the steering mechanism. In some embodiments, a separate cover element 307 may cover the gimbals. The separate cover element 307 may be made of flexible material. In some embodiments, the end effector may be covered by a separate cover that may be rigid or flexible.

Shown in FIG. 13B is a side view of the medical device 300. In some embodiments, the rear (proximal) portion 303 of the adjustable cover is fixed to the base of the medical device. In some embodiments, the distal end 3032 of the rear portion 303 of the cover is fixed to the device's base. The rear portion 303 may be a stationary portion, i.e., it does not move relative to the base of the device during operation of the device. The rear portion of the adjustable cover may be the largest element of the adjustable cover.

Shown in FIG. 13C is a top view of the medical device 300, showing the end effector 304, configured to insert a medical tool 308 into a body of a subject and the adjustable protective cover 302 covering the steering mechanism of the device. In FIG. 13C, the end effector 304 is shown moved and rotated to the left. The movement of the steering mechanism required to position the end effector 304 as shown, caused of the cover elements 309 to move relative to each other, such that several of the cover elements 309 have been inserted further into the adjacent cover element and several of the cover elements have been extended further out of the adjacent cover element, thus maintaining the coverage integrity of the inner components of the device.

Shown in FIG. 13D is a rear view of medical device 300 (with the rear end of the cover omitted), showing portions of a steering mechanism of the device, including movable arm assemblies 314A and 314B. The steering mechanism of the device is covered by the adjustable protective cover 302. Adjustable protective cover 302 is a collapsible cover, configured to extend/expand or collapse, as well as move/rotate sideways in accordance with the movement of the steering mechanism and the consequent movement of the end effector.

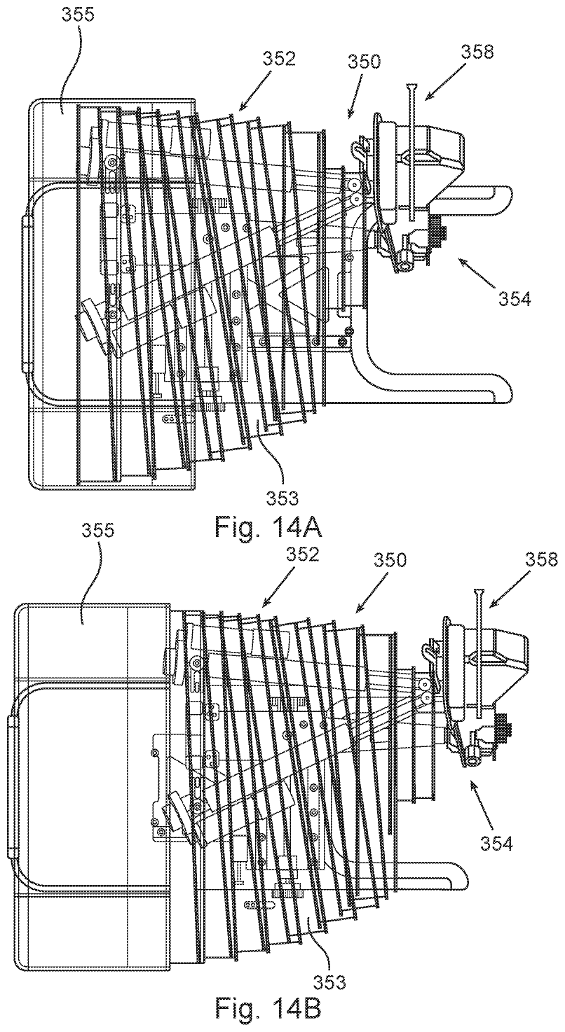

Reference is now made to FIGS. 14A-14B, which illustrate schematic top views of a medical device having a protective cover, according to some embodiments. Shown in FIGS. 14A-14B are top perspective views of an exemplary medical device 350, having an end effector 354, configured to insert a medical tool 358 into a body of a subject. As shown in FIG. 14A, the steering mechanism of the device is covered by an adjustable protective cover 352 (shown in FIG. 14A in semi-transparent view, to reveal the covered steering mechanism). The adjustable protective cover 352 is a collapsible cover, configured to extend or collapse, as well as move sideways in accordance with the movement of the steering mechanism and the consequent movement of the end effector. In some embodiments, at least part of the protective cover 352 may be constructed from partially overlapping consecutive elements 353. The cover elements 353 may have an annular shape. The protective cover 352 in FIG. 14A is shown in a collapsed/retracted position, and curved towards one side of the device, as dictated by the relative position of the steering mechanism elements and the resulting position of the end effector, as well as by the shape and arrangement of the cover elements 353. In FIG. 14B the protective cover 352 (shown in FIG. 14B in semi-transparent view) is shown in an extended/open position, and curved towards one side of the device, as dictated by the relative position of the steering mechanism elements and the resulting position of the end effector, as well as by the shape and arrangement of the cover elements 353. In some embodiments, as shown in FIGS. 14A-14B, a rear portion 355 of the adjustable cover, which may be the largest portion of the cover, may be attached/mounted/fixed to the base of the device. the rear portion 355 may be rigid, i.e., non-collapsible.

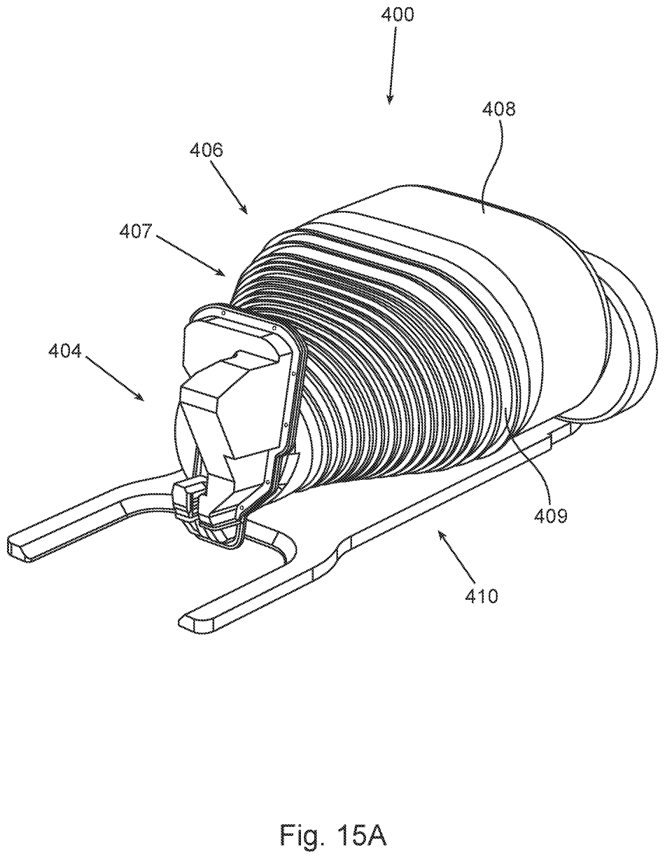

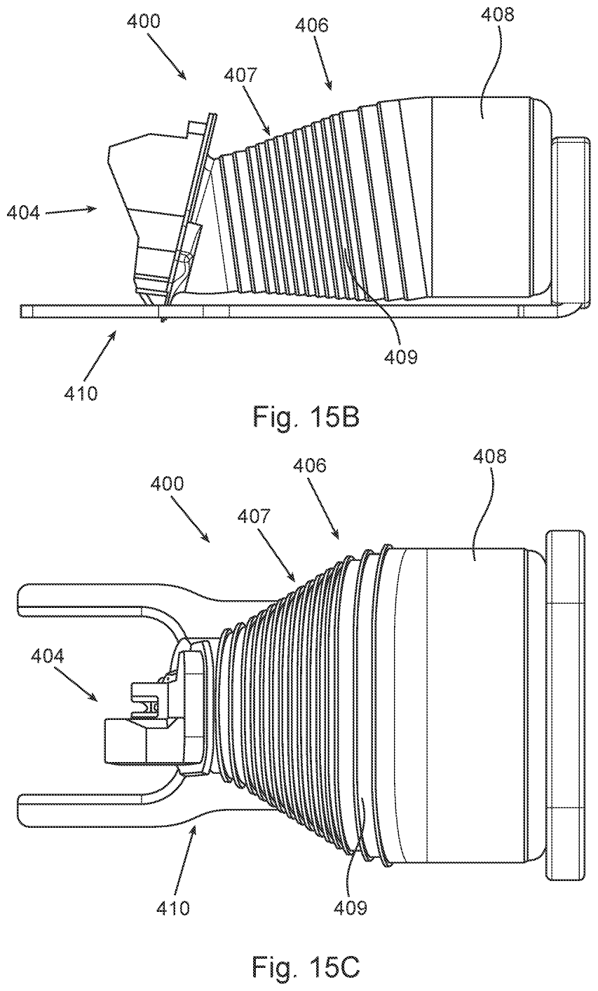

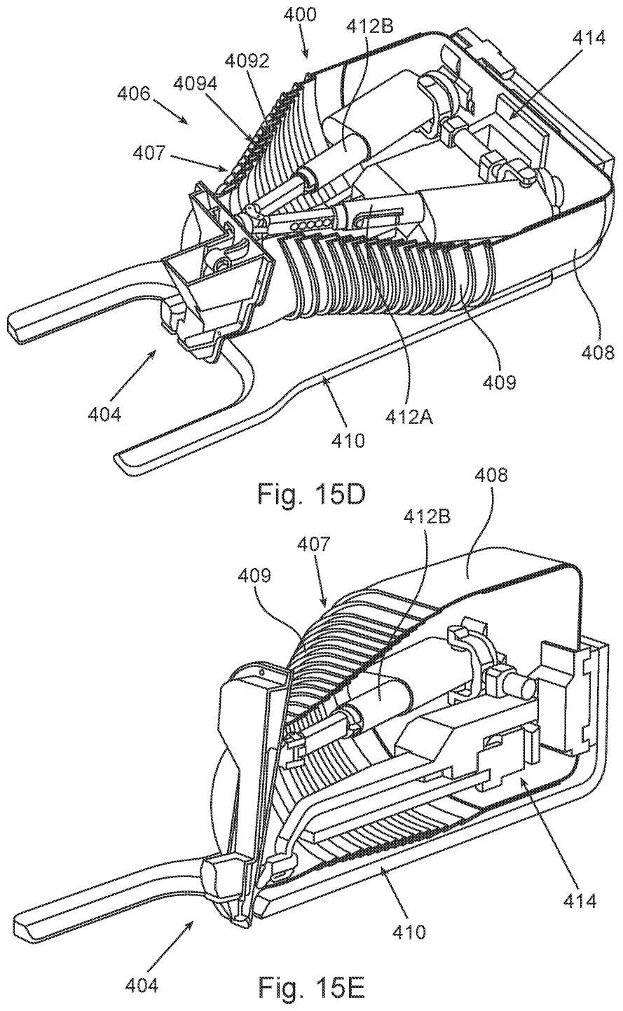

Reference is now made to FIGS. 15A-15E, which illustrate schematic views of flexible adjustable protective covers of elements of the steering mechanism of a medical device, according to some embodiments. Shown in FIG. 15A is a perspective view of a medical device 400, having an end effector 404, and a base 410. The steering mechanism (not shown) of the device is covered by adjustable protective cover 406. Adjustable protective cover 406 is a collapsible (e.g., retractable, telescopic, “scale-like”) cover, configured to at least partially extend/open or collapse/close, as well as move and/or curve/rotate sideways in accordance with the movement of the steering mechanism and the consequent movement of the end effector. In some embodiments, the protective cover may have a conic tube-like shape when the steering mechanism is in its neutral position. In some embodiments, the protective cover may have a rectangular tube-like shape. Adjustable protective cover 406 may include at least two regions/portions: a rear portion 408 and a front portion, for example contracting portion 407. The protective cover may be made of flexible, semi-flexible or rigid material. The contracting portion 407 may be constructed from partially overlapping consecutive elements 409, each element having a smaller size (e.g., diameter or circumference) relative to the preceding element, such that an element may be at least partially inserted into the preceding element, or folded against a preceding element, whereby, the closer the covering element is to the end effector, the smaller the size thereof. The cover elements 409 may have an annular shape. In some embodiments, a flexible and/or sealing material may be disposed between adjacent elements of the cover. The rear portion 408 of the adjustable cover (i.e., the portion that is farthest from the end effector side) may be attached/mounted/fixed to the base of the device and it may have the largest size, compared to the covering elements (“scales”) of the contracting portion. In some embodiments, the rear portion of the adjustable cover is made of one element.

Shown in FIG. 15B is a side view of medical device 400. The steering mechanism (not shown) of the device is covered by adjustable protective cover 406. Adjustable protective cover 406 is configured to at least partially extend/open or collapse/close, as well as, optionally, move/rotate sideways, in accordance with the movement of the steering mechanism. Adjustable protective cover 406 may include at least two regions/portions: a rear portion 408, and a front portion, for example contracting portion 407, wherein the contracting portion includes consecutive elements 409, decreasing in diameter thereof, the closer they are to the end effector.

Shown in FIG. 15C is a top view of medical device 400. In FIG. 15C, the steering mechanism of the device, which is covered by adjustable protective cover 406, is in its neutral position.

Shown in FIG. 15D is a perspective top view of a cross-section of medical device 400. The steering mechanism 414 (including exemplary movable arms 412A-412B)) of the device is covered by adjustable protective cover 406. Adjustable protective cover 406 may include at least two regions/portions: a rear portion 408 and a front portion, for example contracting portion 407, wherein the contracting portion includes consecutive elements 409, decreasing in size/circumference, such that each element is at least partially inserted into the preceding element, whereby, the closer the cover element 409 is to the end effector, the smaller the size thereof. The consecutive elements may be partially overlapping, with the overlapping degree being adjustable, such that the contracting portion 407 is configured to expand or collapse, according to the movement of the steering mechanism. In some embodiments, the cover elements 409 of the contracting portion 407 may comprise constraining members, for example constraining members 4092 and 4094, to limit the movement of the cover elements relative to each other and prevent the cover elements from detaching from one another as the contracting portion 407 contracts/extends. In some embodiments, the constraining members 4092 and 4094 extend along the outer circumferences of the cover element 409.

Shown in FIG. 15E is a perspective side view of a longitudinal cross-section of medical device 400, with the steering mechanism 414, including exemplary movable arms (only arm 412B is shown in FIG. 15E), of the device covered by adjustable protective cover 406.

Implementations of the devices, systems and methods described above may further include any of the features described in the present disclosure, including any of the features described hereinabove in relation to other device, system and method implementations.

The terms “implementation” and “embodiment” are used interchangeably throughout this disclosure.

It is to be understood that the terms proximal and distal as used in this disclosure have their usual meaning in the clinical arts, namely that proximal refers to the end of a device or object closest to the person or machine inserting or using the device or object and remote from the patient, while distal refers to the end of a device or object closest to the patient and remote from the person or machine inserting or using the device or object.

Although some examples used throughout this disclosure relate to an automated insertion device, it is to be understood that the scope of this disclosure is not meant to be limited to adjustable covers for insertion devices, but the disclosed adjustable covers can be used to cover any medical device.

It is also to be understood that although some examples used throughout this disclosure relate to a needle, this is done for simplicity reasons alone, and the scope of this disclosure is not meant to be limited to a needle, but is understood to include any medical tool which is insertable into the subject's body for diagnostic and/or therapeutic purposes, including an introducer, catheter, cannula, port, electrode rod, surgical tool, fluid delivery tool, or any other such insertable tool.

In addition, the terms “subject” and “patient” are used interchangeably throughout this disclosure and may refer to any human or animal undergoing the medical procedure.

In some embodiments, the terms “medical instrument” and “medical tool” may interchangeably be used.

In some embodiments, the terms “cover” and “protective cover” may interchangeably be used.

In some embodiments, the terms “cover portion” and “cover element” may interchangeably be used.

In some embodiments, the terms “left” and “right” are with respect of the point of view of the medical device, i.e., the point of view when looking at the medical device from the back end thereof.

In some embodiments, the term “about” may be used to specify a value of a quantity or parameter to within a continuous range of values in the neighborhood of (and including) a given (stated) value. According to some embodiments, “about” may specify the value of a parameter ±20% of the given value.

In the description and claims of the application, the words “include” and “have”, and forms thereof, are not limited to members in a list with which the words may be associated.

Although particular implementations have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the disclosure as defined by the claims. Other aspects, advantages, and modifications are considered to be within the scope of the following claims. The claims presented are representative of the implementations and features disclosed herein. Other unclaimed implementations and features are also contemplated. Accordingly, other implementations are within the scope of the following claims.

1.-37. (canceled)

38. An adjustable cover for a medical device, the adjustable cover comprising:
one or more stationary cover portions; and
one or more moveable cover portions comprising a plurality of moveable cover elements configured for movement relative to each other and relative to the one or more stationary cover portions,
wherein the one or more stationary cover portions comprise one or more openings, such that the plurality of moveable cover portions are disposed within the one or more openings,
wherein at least one of the plurality of moveable cover elements is configured to move relative to at least one of: (i) an adjacent moveable cover element of the plurality of moveable cover elements and (ii) the one or more stationary cover portions, as one or more components of a driving mechanism and/or a steering mechanism of the medical device move, such that both coverage and proper operation of the driving mechanism and/or the steering mechanism are maintained.

39. The adjustable cover of claim 38, wherein at least two of the plurality of moveable cover elements at least partially overlap.

40. The adjustable cover of claim 38, wherein at least one opening of the one or more openings is a top opening located at a top surface of at least one of the one or more stationary cover portions, wherein the one or more moveable cover portions further comprise a top moveable cover portion disposed within the top opening.

41. The adjustable cover of claim 40, wherein at least one opening of the one or more openings is a front opening located at a front surface of at least one of the one or more stationary cover portions,
wherein the one or more moveable cover portions further comprise a front moveable cover portion disposed within the front opening,
wherein the front moveable cover portion being substantially perpendicular to the top moveable cover portion, and comprises front moveable cover elements,
wherein the front moveable cover elements configured to at least partially overlap with each other such that a left-right movement of an extending arm of the driving mechanism and/or the steering mechanism moves the front cover portion elements, while maintaining coverage of the front opening of the casing.

42. The adjustable cover of claim 38, wherein the plurality of moveable cover elements are configured as a flat frame and configured to slide one over the other.

43. The adjustable cover of claim 38, wherein the one or more stationary cover portions comprises one or more guides disposed on an inner surface thereof, the one or more guides being configured to support and guide the sliding movement of the plurality of moveable cover elements,
wherein each of the one or more guides comprises one or more rails configured to receive an end of at least one of the plurality of moveable cover elements, wherein each moveable cover element of the plurality of moveable cover elements is configured to be supported by two rails oppositely disposed two guides of the one or more guides.

44. The adjustable cover of claim 43, wherein the one or more rails of the one or more guides are arranged in a crosshatch pattern, and configured to arrange the plurality of moveable cover elements in a crosshatch pattern,
wherein the plurality of moveable cover elements are slidable one over the other and may at least partially overlap along two faces of each,
wherein the arrangement of the plurality of moveable cover elements is configured to allow movement in various linear directions, including left-right and/or inward-outward to provide a full coverage of the one or more openings while maintaining full movement range of the steering mechanism.

45. The adjustable cover of claim 38, wherein the one or more moveable cover portions is configured to collapse or expand, according to the movement of the one or more components of the driving mechanism and/or the steering mechanism.

46. The adjustable cover of claim 38, further comprising at least one sealing layer disposed between at least two of the plurality of moveable cover elements.

47. The adjustable cover of claim 38, wherein at least one of the one or more stationary cover portions is configured for attachment to a base of the medical device.

48. The adjustable cover of claim 38, wherein the at least one of the one or more moveable cover portions is moveable by the movement of the one or more components of the driving mechanism and/or the steering mechanism.

49. The adjustable cover of claim 38, wherein the adjustable cover is configured to cover inner components of the medical device.

50. The adjustable cover of claim 38, wherein the medical device is an automated medical device comprising at least four degrees of freedom (DOF); wherein the medical device is an automated insertion medical device configured to insert a medical instrument into a body of a subject, wherein the steering mechanism comprises one or more actuators configured for inserting and steering the medical instrument into and within the body of the patient.

51. The adjustable cover of claim 38, wherein the adjustable cover is configured to at least partially cover and protect portions of the medical device from environmental deposits comprising one or more of: biological fluids, airborne bacteria, humidity, dust, dirt or any combination thereof.

52. An adjustable cover for a medical device, the adjustable cover comprising:
one or more stationary cover portions, at least one of the one or more cover portions comprising one or more openings; and
one or more moveable cover portions disposed within the one or more openings and comprising a plurality of partially overlapping moveable cover elements, the plurality of moveable cover elements being configured for movement relative to each other and relative to the one or more stationary cover portions,
wherein at least one of the plurality of moveable cover elements is configured to move relative to at least one of: (i) an adjacent moveable cover element of the plurality of moveable cover elements and (ii) the one or more stationary cover portions, as one or more components of a steering mechanism of the medical device move, such that both coverage and proper operation of the steering mechanism are maintained.

53. The adjustable cover of claim 52, wherein the one or more openings comprise a top opening located at a top surface of at least one of the one or more stationary cover portions, and the one or more moveable cover portions comprise a top moveable cover portion disposed within the top opening.

54. The adjustable cover of claim 53, wherein the one or more openings further comprises a front opening located at a front surface of the at least one of the one or more stationary cover portions, and the one or more moveable cover portions further comprise a front moveable cover portion disposed within the front opening, wherein the top and front moveable cover portions are configured such that the movement of the one or more components of the steering mechanism of the medical device results in simultaneous movement of at least one moveable cover element of the top moveable cover portion and at least one moveable cover element of the front moveable cover portion.

55. The adjustable cover of claim 52, wherein the one or more stationary cover portions comprises one or more guides disposed on an inner surface thereof, the one or more guides being configured to guide the movement of the plurality of moveable cover elements, wherein each moveable cover element of the plurality of moveable cover elements is configured as a flat frame

56. The adjustable cover of claim 55, wherein each of the one or more guides comprises one or more rails configured to receive an end of at least one of the plurality of moveable cover elements.

57. An automated medical device for insertion of a medical instrument into a body of a subject, the device comprising:
a steering mechanism comprising one or more actuators configured for inserting and steering the medical instrument into and within the body of the patient; and
an adjustable cover comprising:
one or more stationary cover portions; and
one or more moveable cover portions comprising a plurality of moveable cover elements configured for movement relative to each other and relative to the one or more stationary cover portions,
wherein the one or more stationary cover portions comprise one or more openings, such that the plurality of moveable cover portions are disposed within the one or more openings,
wherein at least one of the plurality of moveable cover elements is configured to move relative to at least one of: (i) an adjacent moveable cover element of the plurality of moveable cover elements and (ii) the one or more stationary cover portions, as one or more components of a driving mechanism and/or a steering mechanism of the medical device move, such that both coverage and proper operation of the driving mechanism and/or the steering mechanism are maintained.
